(19) United States
(12) Patent Application Publication
(10) Pub. No.: US 2002/0176876 A1
(43) Pub. Date: Nov. 28, 2002

(54) Topical therapeutic skin care system

(76) Inventors: Dennis H. Harris, Scottsdale, AZ (US); Ronald E. General, Scottsdale, AZ (US)

Correspondence Address: JOSEPH W MOTT, JENNINGS STROUSS & SALMON PLC, 201 EAST WASHINGTON STREET, 11TH FLOOR, PHOENIX, AZ, 85004-2385, US

(21) Appl. No.: 10/053,794

(22) Filed: Jan. 19, 2002

Related U.S. Application Data
(60) Provisional application No. 60/263,826, filed on Jan. 23, 2001.

(51) Int. Cl. A61K 35/80; A61K 35/78; A61K 7/00

(52) U.S. Cl. 424/401; 424/195.17; 424/765; 424/764; 424/744; 424/746

(57) ABSTRACT

A two phase topical therapeutic skin treatment is disclosed, including a first phase composition having antibacterial, anti-inflammatory, humectant, antioxidant and exfoliant ingredients, and a second phase having anti-inflammatory, circulatory enhancement and prolonged moisturizing ingredients.

This application is a continuation-in-part of provisional patent application Ser. No. 60/263,826, filed Jan. 23, 2001.

### BACKGROUND

According to the American Diabetes Association, almost 20,000,000 cases of diabetes have been diagnosed in the United States of America. They estimate that an additional 16,000,000 undiagnosed cases of diabetes will be found within the next three to five years, bringing the total to 36,000,000 cases by 2005. Diabetes is increasing at the incredible rate of 15% per year. Diabetes is now identified as the third leading cause of death in adults.

There are many complications of diabetes, including the following:

1. Diabetic retinopathy leading to visual loss and blindness.

2. Diabetic nephropathy leading to kidney failure.

3. Diabetic neuropathy leading to numbness, abnormal sensations, pain and weakness in the arms and legs.

4. Diabetic feet leading to ulcerations, infections and amputations of the lower extremities.

Diabetes causes a gradual narrowing or occlusion of the microscopically small blood vessels that supply oxygen, nutrients and moisture to the individual cells in many organs, including the skin and peripheral nerves. As the microcirculation to the surface of the feet decreases, many changes occur. Under normal conditions, a constant dynamic balance exists between cells that die and are sloughed off the surface of the skin of the foot and replacement by new cells. The continual decrease in circulation dramatically reduces the amount of moisture and oxygen that can reach the surface layers of skin lining the sole of the feet. This accelerates the rate at which the surface cells die, as well as interfering with the normal elimination of the dead cells in these outer layers, causing them to pile up and become thicker and dryer, often leading to a layer of callus that may reach a thickness of 1/4 inch (6.35 mm) or more.

In addition, the thickening and further drying of the tissues produces stiffening due to the loss of flexibility and elasticity of this layer of callus. Continued walking will then cause cracking that will extend across the entire width of the foot. Due to the dryness of this area, that crack will rapidly deepen to involve the full thickness of the skin, opening a potential avenue for infection leading directly from the outside of the foot to the deeper layers of the skin or even into the muscle below. Since this condition most commonly occurs in conjunction with diabetic neuropathy, the numbness in the feet prevents the patient from becoming aware of these lesions until they reach an advanced stage.

Once infection is established in the deeper layers of the skin or in the muscle, it is extremely difficult to treat. The decreased blood flow to these areas prevents effective levels of oral or injected antibiotics from reaching the site of the infection. Topically applied antibiotics are ineffective in fighting severe or deeper infections. The infection is therefore free to quickly penetrate through the depth of the muscle and subsequently infect the underlying bone, resulting in osteomyelitis. After the osteomyelitis is well established, amputation often becomes the only available treatment. Diabetes is now the leading cause of nontraumatic amputations in the United States and Europe.

Although many lotions and creams to soften the skin have been used, no specific formulation that treats all of the underlying factors of this problem has been available up to this time.

Although the description above is specific for diabetes, a similar process occurs in the elderly patient, producing vascular ulcers that may also lead to amputation or other disability.

Due to the thinning of the ozone layer, longer life span and other factors, skin cancers have become much more common, along with a higher prevalence of warts, moles and liver (aging) spots. Older age is also characterized by a higher incidence of chronic illnesses, leading to bedsores and skin irritation due to incontinence. Advancing age also brings with it oxidative damage to the skin and underlying tissues, resulting in dry, often irritated, skin and wrinkling.

Acne continues to plague adolescent children, requiring the use of over the counter lotions, creams or pads, antibiotics, or high dose Vitamnin A derivative treatments. In spite of all treatment, acne remains a major source ofjuvenile scarring which may have a significant social and psychological impact.

Other common skin diseases, such as psoriasis and eczema, have continued to elude researchers seeking effective treatment modalities.

Therefore, it is the object of the present invention to provide a unique combination of ingredients that will increase superficial circulation, reduce inflammation, speed wound healing, exfoliate dead tissue, reduce the superficial bacterial concentration, stimulate the growth of healthy tissue, clean the surface of the skin, provide antioxidant nutrients to the skin and underlying tissues and provide long lasting moisturizing action to the skin and subcutaneous tissues.

### SUMMARY OF THE INVENTION

This invention overcomes the deficiencies of the prior approaches by combining specific ingredients to accomplish particular therapeutic objectives.

Broadly speaking, the invention consists of two separate formulations which may be used separately or in combination with one another. Both formulations share some common traits. Specifically, they both have significant anti-inflammatory activity, both stimulate and speed the healing process and both confer significant antioxidant activity across the skin barrier. The first formulation, referred to hereinafter as Phase I, also contains an antibacterial portion, a humectant portion and an exfoliant portion. The second formulation, hereinafter referred to as Phase II, contains a circulatory stimulant portion and a tissue moisturizing portion.

The antibacterial action in Phase I is provided by methylparaben, sage extract and triclosan, although other biocytic compounds, such as 8-hydroxyquinoline or similar compounds, could be used instead of, or in addition to triclosan. Ethyl paraben and/or propyl paraben could also be used in addition to or instead of methylparaben. Other compounds, such as tincture of iodine or Melaleuca alternifolia, could also be incorporated.

The anti-inflammatory action and stimulation of the healing process are well known and often described characteristics of aloe vera, which has been incorporated into many products designed for healing skin. Dexapanthenol, sage extract and chamomile extract have also demonstrated excellent anti-inflammatory properties. The addition or substitution of other anti-inflammatory compounds, such as alphabisabolol or chemazulene could enhance or modify the level of anti-inflammatory activity.

The humectant action may be provided by glycerin, glyceryl monostearate or propylene glycol, as well as any other form of glycerol, although xylenol, ethylene glycol or sorbitol could also be utilized as effective humectants.

The antioxidant activity is related to the incorporation of Rose Hips Oil, an abundant natural source of Vitamin C. Antioxidant activity can be enhanced by the addition or substitution of other forms of Vitamin C, Vitamin E in various forms, pycnogenol, and/or glutathione.

The synergistic action of seaweed extract and glycerin produce a nonirritating, but extremely potent exfoliant action.

In Phase II, the anti-inflammatory activity and stimulation of healing is related to the presence of aloe vera and dexapanthenol, as noted above. The same additions and/or modifications noted above in relation to Phase I would be applicable in Phase II.

The circulatory enhancement is due to the inclusion of a superficial vasodilator, such as nutmeg oil, cinnamon oil, cinnamaldehyde, nicotinic acid or its derivatives, yohimbine or glycerine.

Prolonged tissue moisturization is achieved by the synergistic effect of glycerin, aloe vera and sorbitol.

By utilizing this unique combination of ingredients and adding additional ingredients to enhance specific biological effects, these formulations can be used, either separately or in combination, for the prevention and treatment of a wide variety of skin conditions, including diabetic feet, vascular insufficiency, vascular and pressure ulcers, dry skin, skin irritation, first and second degree bums, open skin tears and abrasions, wrinkles, precancerous skin lesions, moles, warts, liver spots, acne, psoriasis, eczema and other similar skin lesions. Phase I, used alone, is an extremely effective antibacterial waterless hand cleanser. Either formulation may include a cosmetic surfactant and a cosmetic preservative.

Varying ratios of the different components of each formulation are contemplated depending upon the condition to be treated. Some specific embodiments will be utilized in the following detailed description of the invention for illustrative purposes. Furthermore, the ingredients may be varied within a wide range, or additional ingredients added, in order to adapt these formulations for specific delivery systems, such as spray, lotion, cream, gel or ointment.

### DETAILED DESCRIPTION OF THE INVENTION

Many clinical experiments have proven that the primary elements leading to foot complications and subsequent amputation in diabetic patients are the thickening and dehydration of dead skin cells on the sole of the foot associated with reduced microcirculation and infection of the surface of the foot and underlying tissues. The present invention corrects these deficiencies in the following manner: 1) reducing the bacterial population on the surface of the foot; 2) exfoliating the accumulated layers of dead skin cells; 3) stimulating the circulation to the surface of the foot; and 4) providing long-lasting moisterization of the skin.

These characteristics, along with others available when the formula is modified, make this invention an effective treatment modality for many other skin conditions, including, but not limited to, vascular insufficiency, vascular ulcers, pressure ulcers (bedsores), dry skin, skin irritation due to perspiration or urinary or fecal incontinence, first and second degree burns, open skin tears and abrasions, wrinkles, precancerous skin lesions, moles, warts, liver spots, acne, psoriasis, and eczema.

The preferred embodiment is as follows, with all percentages expressed by weight:

Therapeutic Lotion For Diabetic Feet

Phase I

| Ingredient | Percentage |
|---|---|
| Purified Water | 60.0 |
| Aloe Vera Gel | 25.7 |
| Glycerin | 5.0 |
| Polyvinol Crystalline | 8.0 |
| Seaweed Extract | 0.2 |
| Triclosan | 0.2 |
| Pantothenic Acid | 0.2 |
| Sage Extract | 0.2 |
| Chamomile Extract | 0.2 |
| Rose Hips Extract | 0.2 |
| Methylparaben | 0.1 |

Phase II

| Ingredient | Percentage |
|---|---|
| Glycerin | 91.0 |
| Aloe Vera Gel | 5.0 |
| Sorbitol | 3.6 |
| Vitamin B5 | 0.2 |
| Nicotinic Acid | 0.2 |

A small amount of Phase I may be poured into the hand and applied directly to the affected area. The product may be rubbed over the entire area to be treated in order to apply a thin layer over the area, stopping the application when the lotion dries to the point of tackiness (or stickiness). The drying process may then be allowed to finish, permitting the exfoliative process to proceed. When Phase I is dry, a damp cloth may be used to continuously, but gently, remove Phase I until no additional loose dead skin cells are able to be removed. A small amount of Phase II is then applied to the entire affected area utilizing a gentle massaging technique. Phase II may be left in place. If desired, any excess of Phase II may be removed after several minutes.

In Phase I, 60% purified water by weight was used, as it allowed complete blending of all other ingredients while maintaining an ideal consistency. 25.7% aloe vera gel was used, as it provided an acceptable level of anti-inflammatory effect and stimulation of the healing process without a greasy or oily feeling. 8% polyvinol crystals was utilized to increase the exfoliative effect of the product without leaving a sticky feeling after drying. 5% glycerin was used to obtain the correct humectant action. Increased amounts of glycerin tends to impair the exfoliant activity. 0.2% seaweed extract provides improved exfoliation while stabilizing the correct viscosity. 0.2% triclosan was used, as it is the maximum amount allowed by the United States Food and Drug Administration and is needed to achieve the antibacterial action required. A lower dosage would decrease the effectiveness. 0.2% Vitamin B5 (dexapanthenol or pantothenic acid) was used to promote the healing process; an the amount of pantothenic acid is kept low because it can produce local irritation. 0.2% sage extract was used to additionally stimulate the healing process and produce an anti-inflammatory effect, as well as adding to the antibacterial activity. Higher percentage of sage extract may be locally irritating, while a lower amount of sage extract can detract from the healing process and the antibacterial activity. 0.2% chamomile extract was used to supplement the anti-inflammatory effect and speed the healing process and 0.2% rose hips extract was utilized to produce a significant degree of antioxidant activity. Both of these ingredients may cause localized skin irritation unless concentrations are kept relatively low. 0.1% methylparaben was used to increase the range and efficiency of the antibacterial component of the product, as well as stabilizing the product to increase the shelf life. Too little methylparaben would result in a decreased shelf life and narrower range of antibacterial activity, while an excess of methylparaben could introduce some toxicity factors.

In Phase II, 91% glycerin was used to maximize the humectant activity and the skin moisturization. Too little glycerin would impact on the ability of the product to attract and retain an adequate amount of moisture to achieve the objective of this phase of the product, while an excess amount of glycerin would excessively increase the oiliness of the product. 5% aloe vera gel was used to ensure an additional amount of moisturization of the tissues. Too little aloe vera gel would reduce the moisture retaining ability of the formula, as well as less tissue healing ability, while too much aloe vera gel would result in a greasy or oily feeling on the skin. 3.6% sorbitol was used to further improve the tissue moisture retention. Too little sorbitol would decrease the ability of the product to attract and retain moisture, while too much sorbitol would impart a greasy or oily feeling to the skin. 0.2% pantothenic acid was used to achieve a good level of anti-inflammatory effect, as well as speeding up the healing process. Too little pantothenic acid would be less effective in improving the healing process, while too much of this component would cause skin irritation. 0.2% nicotinic acid was used to obtain excellent superficial peripheral vasodilitation. Too little of this component would result in inadequate vasodilitation, while an excess amount of nicotinic acid would be extremely irritating.

A more generalized embodiment is as follows, with all percentages expressed by weight:

Therapeutic Lotion For Diabetic Feet

Phase I

| Ingredient | Percentage |
|---|---|
| Purified Water | 1.0-60.0 |
| Aloe Vera Gel | 0.5-50.0 |
| Polyvinol Crystals | 2.0-15.0 |
| Glycerin | 5.0-50.0 |
| Seaweed Extract | 0.1-3.0 |
| Triclosan | 0.05-0.2 |
| Pantothenic Acid | 0.1-1.0 |
| Sage Extract | 0.1-5.0 |
| Chamomile Extract | 0.1-5.0 |
| Rose Hips Extract | 0.1-5.0 |
| Methylparaben | 0.05-0.25 |

Phase II

| Ingredient | Percentage |
|---|---|
| Glycerin | 25.0-91.0 |
| Aloe Vera Gel | 0.5-50.0 |
| Sorbitol | 0.1-5.0 |
| Pantothenic Acid | 0.1-1.0 |
| Nicotinic Acid | 0.05-2.0 |

Phase I, the range of purified water specified accommodates the variety of dilutions that are required for different levels of skin texture and oiliness. The range of aloe vera gel allows for its use in various combinations with sage extract, chamomile extract and dexapanthenol without producing any excess oiliness on the skin. The range of polyvinol crystals used allows for maximum exfoliation without producing excess stickiness or skin irritation. The range of glycerin used in this phase was designed to achieve the desired humectant effect without producing excess moistening of the skin, which may lead to maceration. The range of seaweed extract used allows for varying degrees of exfoliation under different disease states, as well as regulating the viscosity of the mixture. The range of triclosan used allows for variable levels of antibacterial activity. The range of pantothenic acid used permits variable combinations with sage extract, aloe vera gel and chamomile extract to provide optimal stimulation of healing and anti-inflammatory activity while preventing skin irritation from occurring. The range of sage extract used permits variable combinations with pantothenic acid, aloe vera gel and chamomile extract to provide optimal stimulation of healing and anti-inflammatory activity and varying combinations with triclosan and methylparaben to achieve a broader antibacterial range while preventing skin irritation from occurring. The range of chamomile extract used permits variable combinations with pantothenic acid, aloe vera gel and sage extract to provide optimal stimulation of healing and anti-inflammatory activity while preventing skin irritation from occurring. The range of rose hips extract used provides the necessary range of antioxidant activity to maintain skin integrity while preventing irritation of the skin. The range of methylparaben used permits varying combinations with triclosan and sage extract to provide the optimal level and broadest range of antibacterial activity without reaching toxic levels. This will also provide the longest shelf life within that range.

In Phase II, the range of glycerin used is designed to provide the optimal humectant effect to moisturize the skin and underlying tissues in a variety of skin conditions. Too little glycerin would not correct the skin dryness, and too much glycerin would impart an oily or greasy feel to the skin. The range of aloe vera gel used allows further improvement of the ability of the product to attract and retain moisture when used in varying combination with glycerin and sorbitol, while maintaining the ability of the formula to stimulate and speed the healing process. Too little aloe vera gel would decrease the tissue moisturization, and too much aloe vera gel would result in increased oiliness of the product. The range of sorbitol was selected to optimize the ability of the formula to provide the desired tissue moisture retention, particularly when used in different combinations with glycerin and aloe vera gel. Sorbitol also is utilized to regulate the viscosity of the product. Too little sorbitol would impact upon the duration of tissue moisturization and produce reduced product viscosity, while too much sorbitol would produce an oily or greasy product. The range of pantothenic acid used permits variable combinations with aloe vera gel to provide the optimal level of healing stimulation and anti-inflammatory activity needed for different skin conditions. Too little pantothenic acid results in slower healing, while an excess of pantothenic acid would produce skin irritation. The range of nicotinic acid utilized in this formula is very critical in producing just the desired amount of vasodilitation of the superficial vessels. Too little nicotinic acid will result in continued circulatory depletion, while too much nicotinic acid will lead to excess vasodilitation, producing excessive metabolic demands on damaged tissues with the possibility of tissue death occurring.

Phase I of this embodiment, the antibacterial action is provided by triclosan, sage extract and methylparaben. Stimulation of the healing process and anti-inflammatory activity are due to aloe vera, sage extract, Dexapanthenol (Vitamin B5), and Chamomile extract. Glycerin acts a humectant. Rose Hips extract provides a potent antioxidant due to its generation of Vitamin C, the best of the antioxidants. Powerful, but nonirritating, exfoliation is accomplished through the synergistic action of seaweed extract and glycerin.

In Phase II of this embodiment, stimulation of the healing process and anti-inflammatory activity are due to the presence of aloe vera and Dexapanthenol (Vitamin B5) as described above. Enhancement of circulation to the skin surface and underlying tissues is provided by nicotinic acid, a well studied superficial vasodilator. Long lasting tissue moisturization is produced by the synergistic effect of glycerin, aloe vera and sorbitol.

Phase I can be used alone as an extremely effective waterless, skin cleanser. When applied in a thin film, Phase I dries into a sheet-like layer. During the drying process, the seaweed extract and glycerin loosen the dead skin cells as the first part of the exfoliation process. The hands are then rubbed together vigorously. During the rubbing and flaking away of Phase I, the skin is massaged, microcirculation is stimulated, skin temperature is increased and dead skin cells are exfoliated along with the removal of any dirt, grease or other skin contaminants. Antibacterial activity is provided by the combination of triclosan, sage extract, melaleuca alternifolia and methylparaben. This process ensures that the skin is clean, soft, free of dead cells, radiant, invigorated and healthy.

The preferred embodiment is as follows, with all percentages expressed by weight:

| Ingredient | Percentage |
|---|---|
| Purified Water | 38.0 |
| Aloe Vera Gel | 25.7 |
| Polyvinol Crystals | 8.0 |
| Glycerin | 5.0 |
| Seaweed Extract | 0.2 |
| Triclosan | 0.2 |
| Pantothenic Acid | 0.2 |
| Sage Extract | 0.2 |
| Chamomile Extract | 0.2 |
| Rose Hips Extract | 0.2 |
| Methylparaben | 0.1 |
| Ethyl Alcohol | 20.0 |
| Melaleuca Alternifolia | 0.5 |
| Vitamin E Acetate | 0.5 |
| Vitamin A Palmitate | 0.5 |

The invention, as portrayed in the embodiment of Phases I and II above, is ideal for the treatment of diabetic feet, vascular insufficiency, vascular ulcers, dry skin, chronic cracking of the skin of the hands and/or feet and removal of precancerous skin lesions.

For the treatment of acne, psoriasis and eczyma, modification of Phase I would include the addition of other biocytic compounds, such as 8-hydroxyquinoline and/or Melaleuca alternifolia, in order to achieve maximum antibacterial activity. Phase II would be modified to decrease the viscosity and density, and would also require the addition of Vitamin A in substantial quantities.

The preferred embodiment is as follows, with all percentages expressed by weight:

Phase I

| Ingredient | Percentage |
|---|---|
| Purified Water | 39.5 |
| Aloe Vera Gel | 25.7 |
| Polyvinol Crystals | 8.0 |
| Glycerin | 5.0 |
| Seaweed Extract | 0.2 |
| Triclosan | 0.2 |
| Pantothenic Acid | 0.2 |
| Sage Extract | 0.2 |
| Chamomile Extract | 0.2 |
| Rose Hips Extract | 0.2 |
| Methylparaben | 0.1 |
| Ethyl Alcohol | 20.0 |
| Melaleuca Alternifolia | 0.5 |

Phase II

| Ingredient | Percentage |
|---|---|
| Glycerin | 85.0 |
| Aloe Vera Gel | 5.0 |
| Sorbitol | 3.6 |
| Pantothenic Acid | 0.2 |
| Nicotinic Acid | 0.2 |
| Vitamin E Acetate | 0.5 |
| Vitamin A Palmitate | 0.5 |
| White Willow Extract | 5.0 |

A modification of Phase I can also be utilized as a stand-alone treatment for acne, psoriasis or eczema. The preferred embodiment is as follows, with all percentages expressed by weight:

Phase I

| Ingredient | Percentage |
|---|---|
| Purified Water | 38.0 |
| Aloe Vera Gel | 25.7 |
| Polyvinol Crystals | 8.0 |
| Glycerin | 5.0 |
| Seaweed Extract | 0.2 |
| Triclosan | 0.2 |
| Pantothenic Acid | 0.2 |
| Sage Extract | 0.2 |
| Chamomile Extract | 0.2 |
| Rose Hips Extract | 0.2 |
| Methylparaben | 0.1 |
| Ethyl Alcohol | 20.0 |
| Melaleuca Alternifolia | 0.5 |
| Vitamin E Acetate | 0.5 |
| Vitamin A Palmitate | 0.5 |

Phase II can also be used alone as a medical and/or cosmetic skin moisturizer. This formula is unique inasmuch as the combination of ingredients provides up to 12 to 24 hours of moisture retention, whereas all other moisturizers provide only 2 to 3 hours of moisture retention. Two formulations are required to meet the needs of those individuals with normal or dry skin and those with oily skin.

The preferred embodiment for normal to dry skin is as follows, with all percentages expressed by weight:

Phase II

| Ingredient | Percentage |
|---|---|
| Glycerin | 91.0 |
| Aloe Vera Gel | 5.0 |
| Sorbitol | 3.6 |
| Pantothenic Acid | 0.2 |
| Nicotinic Acid | 0.2 |

The preferred embodiment for oily skin is as follows, with all percentages expressed by weight:

Phase II

| Ingredient | Percentage |
|---|---|
| Glycerin | 90.0 |
| Aloe Vera Gel | 5.0 |
| Sorbitol | 3.6 |
| Pantothenic Acid | 0.2 |
| Nicotinic Acid | 0.2 |
| Polysorbate | 1.0 |

In addition to the other applications, a modification of Phase II can also be used as a male and female sexual moisturizer and sexual stimulant. The preferred embodiment for this use is as follows, with all percentages expressed by weight:

Phase II

| Ingredient | Percentage |
|---|---|
| Glycerin | 86.2 |
| Aloe Vera Gel | 5.0 |
| Sorbitol | 3.6 |
| Pantothenic Acid | 0.2 |
| Yohimbine | 5.0 |

Many alterations may be made by those having ordinary skill in the art without departing from the spirit and scope of the invention. Although the present invention has been described with reference to preferred embodiments, numerous modifications and variations can be made, some of which were described above, and the results will still come within the scope of the invention. Said modifications and variations were put forth only by way of example and not as a limitation to the scope of our invention as set forth in the objects thereof and in the appended claims.

We claim:

1. A topical therapeutic skin treatment composition comprising a first phase comprising an effective amount of an antibacterial ingredient, an effective amount of an anti-inflammatory ingredient, an effective amount of a humectant ingredient, an effective amount of an antioxidant ingredient, and an effective amount of an exfoliant ingredient; and a second phase comprising an effective amount of an anti-inflammatory ingredient, an effective amount of a circulatory enhancement ingredient, and an effective amount of a prolonged moisturization ingredient.

2. A topical therapeutic skin treatment composition comprising a first phase comprising by weight from 1 to 60 percent purified water, 0.5 to 50 percent aloe vera gel, 2 to 15 percent polyvinol crystals, 5 to 50 percent glycerin, 0.1 to 3 percent seaweed extract, 0.05 to 0.2 percent triclosan, 0.1 to 1 percent pantothenic acid, 0.1 to 5 percent sage extract, 0.1 to 5 percent chamomile extract, 0.1 to 5 percent rose hips extract, and 0.05 to 0.25 percent methylparaben, and a second phase comprising by weight from 25 to 91 percent glycerin, 0.5 to 50 percent aloe vera gel, 0.1 to 5 percent sorbital, 0.1 to 1 percent pantothenic acid, and 0.05 to 2 percent nicotinic acid.

3. The skin treatment composition of claim 2 comprising about 60 percent purified water, about 25.7 percent aloe vera gel, about 5 percent glycerin, about 8 percent polyvinol crystals, about 0.2 percent seaweed extract, about 0.2 percent triclosan, about 0.2 percent pantothenic acid, about 0.2 percent sage extract, about 0.2 percent chamomile extract, about 0.2 percent rose hips extract, and about 0.1 percent methylparaben in the first phase; and about 91 percent glycerin, about 5 percent aloe vera gel, about 3.6 percent sorbitol, about 0.2 percent patothenic acid, and about 0.2 percent nicotinic acid in the second phase.

4. The skin treatment composition of claim 1 wherein the first phase further includes an effective amount of biocytic component to achieve maximum antibacterial activity, and the second phase further includes a Vitamin A carrying ingredient.

5. A topical therapeutic skin treatment composition comprising an effective amount of an antibacterial ingredient, an effective amount of an anti-inflammatory ingredient, an effective amount of a humectant ingredient, an effective amount of an antioxidant ingredient, and an effective amount of an exfoliant ingredient.

6. The composition of claim 5 further comprising a topical Vitamin A carrying ingredient.

7. A topical therapeutic skin treatment composition comprising an effective amount of an anti-inflammatory ingredient, an effective amount of a circulatory enhancement ingredient, and an effective amount of a prolonged moisturization ingredient.

8. The composition of claim 7 wherein the anti-inflammatory ingredient includes aloe vera gel and pantothenic acid, the circulatory enhancement ingredient is nicotinic acid, and the moisturizing ingredient includes sorbital and glycerin.

9. The composition of claim 7 further including an effective amount of yohimbine for circulatory enhancement.

10. A method of treating skin conditions topically comprising the steps applying a first phase composition comprising an effective amount of an antibacterial ingredient, an effective amount of anti-inflammatory ingredient, an effective amount of a humectant ingredient, an effective amount of an antioxidant ingredient, and an effective amount of an exfoliant ingredient; allowing the composition to dry; removing the first phase composition; applying a second phase composition comprising an effective amount of an anti-inflammatory ingredient, an effective amount of a circulatory enhancement ingredient, and an effective amount of a prolonged moisturization ingredient; and leaving the second phase composition in place.
